(19) United States
(12) Patent Application Publication
(10) Pub. No.: US 2023/0042047 A1
(43) Pub. Date: Feb. 9, 2023

(54) SKIN CARE DEVICE

(71) Applicant: LG ELECTRONICS INC., Seoul (KR)

(72) Inventors: Hyunchul KIM, Seoul (KR); Kyoungtae KIM, Seoul (KR); Yunhee KU, Seoul (KR); Rayoung PARK, Seoul (KR); Dongwon KIM, Seoul (KR)

(73) Assignee: LG ELECTRONICS INC., Seoul (KR)

(21) Appl. No.: 17/788,191

(22) PCT Filed: Dec. 31, 2019
(86) PCT No.: PCT/KR2019/018784

§ 371 Date: Jun. 22, 2022

(30) Foreign Application Priority Data
Dec. 23, 2019 (KR) 10-2019-0173372

(51) Int. Cl. A61N 1/32; A61N 1/04; A61N 1/36; A61L 2/10; A61L 2/26; A61L 2/24; A45D 44/00

(52) U.S. Cl. CPC A61N 1/328; A61N 1/0408; A61N 1/36034; A61L 2/10; A61L 2/26; A61L 2/24; A45D 44/00; A61L 2202/11; A61L 2202/14; A45D 2200/205

(57) ABSTRACT

A skin care device can include a body including a battery and a grip electrode electrically connected to the battery, and a head connected to one end of the body. The head includes a contact electrode electrically connected to the battery and configured to come in contact with a skin of a user, and at least one ultraviolet light source disposed inside the head to face the contact electrode. Further, the contact electrode includes a fastening part fastened to the head and having at least one hole formed therein to correspond to the at least one ultraviolet light source, and a plurality of contact protrusions protruding from a top surface of the fastening part.

### TECHNICAL FIELD

The present disclosure relates to a skin care device, and more particularly, to a device for managing skin of various body parts such as a scalp and the like.

### BACKGROUND ART

Modern people recognize their appearance as an important factor to express and inform the others, thereby investing many costs and time to the appearance care in order to overcome the drawbacks to the appearance.

Among them, skin care aims to maintain clean and smooth skin without blemishes, and especially, the most interest in skin care of the face among body parts is formed. Therefore, people want to keep their skin clean by getting massages for facial skin care, applying functional makeup products, or using various cleansing products.

Among them, in the case of face washing using a cleansing product, there may be concerns that the cleansing product may not be evenly delivered to the skin or that bacterial infection by the hands and the like may be caused. In particular, it may be difficult to effectively eliminate wastes such as fine dust having penetrated into the skin by face washing.

On the other hand, hair loss is caused by various causes such as environmental, genetic, mental, and physical, and various technologies or treatments such as administration of functional products for hair loss prevention or treatment, low-output laser therapy and the like are being studied, and measures to alleviate or treat hair loss through scalp care are also important.

### DISCLOSURE

### Technical Task

One technical task of the present disclosure is to provide a skin care device capable of effectively eliminating wastes having penetrated into skin.

Another technical task of the present disclosure is to provide a skin care device capable of maintaining cleanliness in a portion in contact with the skin.

Further technical task of the present disclosure is to provide a skin care device having an ergonomic structure for convenient use and effective contact with skin.

### Technical Solutions

A skin care device according to an embodiment of the present disclosure may include a contact electrode electrically connected to a battery and coming in contact with a skin, and the contact electrode may form a polarity to discharge waste in the skin when coming in contact with the skin.

The skin care device may include at least one ultraviolet light source disposed inside the head to face the contact electrode. The contact electrode may include a fastening part fastened to the head and having at least one hole formed therein to correspond to the at least one ultraviolet light source and a plurality of contact protrusions protruding from a top surface of the fastening part.

The skin care device may further include a cap detachably attached to the head, and the cap may include a light reflecting part formed on at least one portion of an inner surface of a cap body.

According to an embodiment, a surface of the light reflecting part may form a non-planar or irregular surface.

The head may further include a cap mounting detection sensor. A processor of the skin care device may drive the at least one ultraviolet light source for a preset time based on a detection result of the cap mounting detection sensor.

According to an embodiment, the cap mounting detection sensor may include a hall sensor and the cap may further include a magnet.

A plurality of the contact protrusions form different protrusion heights based on distances spaced apart from a center of a top surface of the fastening part, respectively.

According to an embodiment, a protrusion height of a first contact protrusion spaced apart from the center by a first distance may be equal to or smaller than that of a second contact protrusion spaced apart from the center by a second distance longer than the first distance.

According to an embodiment, wherein a protrusion height of each of a plurality of the contact protrusions may increase in proportion to a distance spaced apart from the center.

According to an embodiment, the skin care device may control a current modulator to alternately form (+) polarity and (−) polarity in the contact electrode for a preset time from a time point of contacting the contact electrode with the skin.

According to an embodiment, the skin care device may control the current modulator to form (−) polarity in the contact electrode for at least one period for a preset time from a time point of contacting the contact electrode with the skin.

A method of controlling a skin care device according to an embodiment of the present disclosure may include based on contacting a contact electrode with a skin, applying a current according to a first pattern to the contact electrode for a preset time from a contact initiation time point and after a lapse of the preset time, applying a current according to a second pattern different from the first pattern to the contact electrode, wherein when the current according to the first pattern is applied, (−) polarity may be formed in the contact electrode for at least one period.

According to an embodiment, when the current according to the second pattern is applied, the (−) polarity may not be formed in the contact electrode.

### ADVANTAGEOUS EFFECTS

According to an embodiment of the present disclosure, a skin care device includes a contact electrode which comes in contact with a skin to form a prescribed polarity, thereby effectively eliminating wastes present on the surface or inside of the skin based on the reverse iontophoresis principle.

In addition, the skin care device provides sterilization function for the contact electrode through at least one ultraviolet light source, thereby maintaining cleanliness of the contact electrode in direct contact with the skin. Accordingly, it may effectively prevent side effects such as deterioration of the skin condition due to bacteria remaining on the contact electrode.

In addition, as a light reflection unit is formed on a cap detachable from a head of the skin care device, an ultraviolet light source may be provided to the head. That is, since the cap may not be provided with an ultraviolet light source and a configuration for supplying power, the structure of the cap may be simplified and other additional devices such as a cradle for sterilizing the contact electrode are not required. As a result, the configuration of the entire package of the skin care device may be simplified.

In addition, since a plurality of contact protrusions are formed on the contact electrode, contact between the skin and the contact electrode can be facilitated when the skin care device is used for a part where hair, etc. is present.

In addition, since the protrusion height of each of a plurality of the contact protrusions is formed to become higher as the corresponding contact protrusion gets far away from the center of a fastening part of the contact electrode, all the contact protrusions may effectively come in contact with a skin when coming in contact with the skin having a curved surface such as a scalp. Accordingly, the care (cleansing) effect provided by the skin care device may be maximized.

Furthermore, the skin care device is driven according to current characteristics that can maximize the elimination of waste inside the skin, thereby maximizing an elimination rate of the waste over the amount of power consumption.

### DESCRIPTION OF DRAWINGS

FIG. 1 is a perspective diagram of a skin care device according to one embodiment of the present disclosure.

FIG. 2 is one side view of a skin care device according to one embodiment of the present disclosure.

FIG. 3 is one schematic cross-sectional diagram of a skin care device.

FIG. 4 is a block diagram schematically showing a control configuration of a skin care device.

FIG. 5 is a diagram to describe an embodiment related to sterilization of a contact electrode provided to a head of a skin care device.

FIGS. 6 to 7B are diagrams to describe an embodiment related to a plurality of contact protrusions included in a contact electrode.

FIG. 8 is a diagram to describe the operation principle of a skin care device according to one embodiment of the present disclosure.

FIG. 9 and FIG. 10 are diagrams to describe a cleansing effect according to the characteristic of a current applied to a contact electrode of a skin care device.

### BEST MODE FOR DISCLOSURE

Description will now be given in detail according to exemplary embodiments disclosed herein, with reference to the accompanying drawings. For the sake of brief description with reference to the drawings, the same or equivalent components may be provided with the same reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, when an element is referred to as being “directly connected with” another element, there are no intervening elements present.

A singular representation may include a plural representation unless it represents a definitely different meaning from the context.

Terms such as “include” or “has” are used herein and should be understood that they are intended to indicate an existence of several components, functions or steps, disclosed in the specification, and it is also understood that greater or fewer components, functions, or steps may likewise be utilized.

Hereinafter, embodiments of the present disclosure will be described in detail with reference to the accompanying drawings in the present specification.

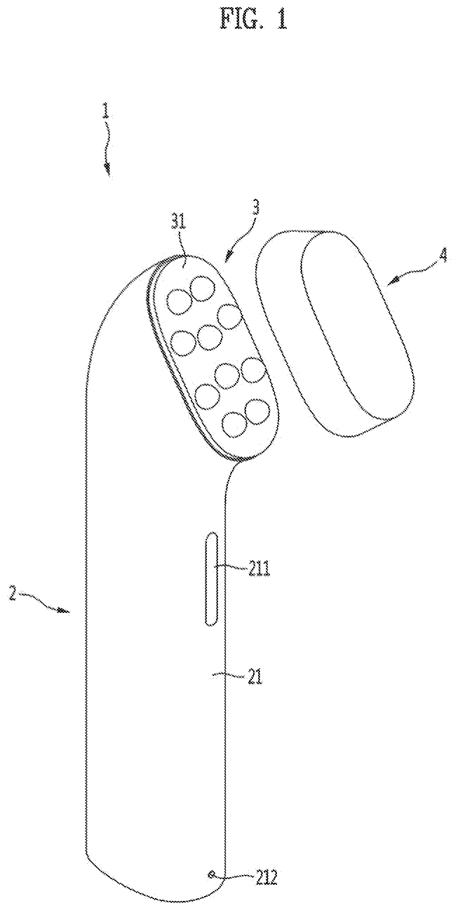

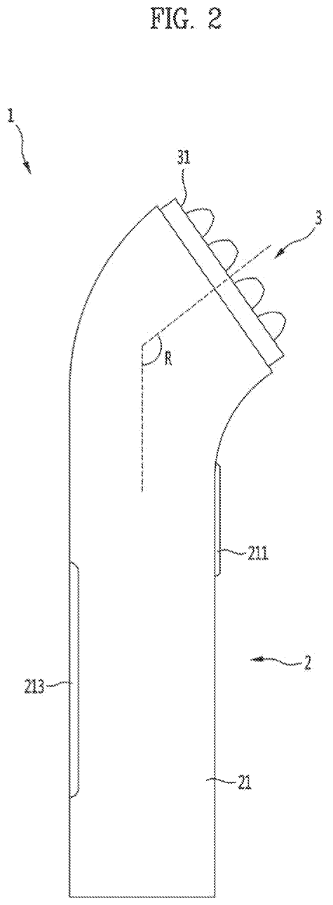

FIG. 1 is a perspective diagram of a skin care device according to one embodiment of the present disclosure, and FIG. 2 is one side view of the skin care device according to one embodiment of the present disclosure.

Referring to FIG. 1 and FIG. 2, a skin care device 1 according to one embodiment of the present disclosure may include a device configured to eliminate wastes of skin based on the reverse iontophoresis technique. Specifically, the skin care device 1 may be a device of eliminating wastes having a different polarity (e.g., (−) polarity) existing on a skin surface or inside a skin (e.g., extracellular fluid, intercellular substance, etc.) by discharging the wastes from the skin in a manner that a contact electrode 31 forming a prescribed polarity (e.g., (+) polarity) comes in contact with user's skin.

Such a skin care device 1 may include a body 2, a head 3, and a cap 4.

The body 2 may have a shape that facilitates a contact electrode 31 of the head to come in contact with a user's skin in a manner of being held by a user's hand. For example, a case 21 forming an exterior of the body 2 is configured to have a cylindrical shape in part at least, thereby facilitating a user to grip the case 21 by a hand.

In addition, in order to facilitate the head 3 to come in contact with a skin while a user grips the body 2, the head 3 may be configured in a manner of being extended from one end of the body 2 to form a prescribed angle R. For example, the angle R may correspond to one of angles between 90° and 180°. Preferably, the angle R may range between 100° and 150°. For example, the angle R may include 125°, by which the angle is non-limited.

At least one input interface (e.g., at least one button 211) for settings of ON/OFF or an operation mode of the skin care device 1 and the like may be provided to the body 2. In addition, at least one hole 212 for external communication with a microphone and/or speaker disposed inside the case 21 may be formed in a prescribed position of the case 21.

In addition, a grip electrode 213, which comes in contact with a user's hand when the user grips the body 2 by a hand, may be provided to the body 2. To come in contact with a user's hand, the grip electrode 213 may be provided to a prescribed position of the case 21 to be exposed therefrom. The grip electrode 213 may be formed of conductive metal such as Stainless Use Steel (SUS), aluminum, etc.

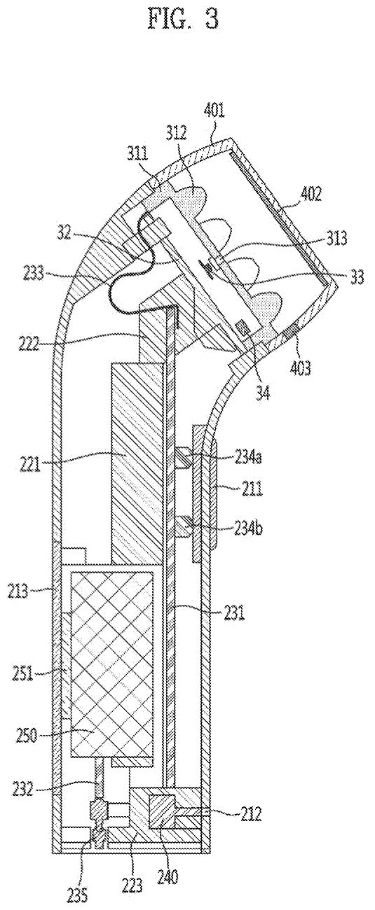

As the grip electrode 213 comes in contact with the user's hand and the contact electrode 31 comes in contact with a user's skin (e.g., scalp, etc.), a closed circuit that connects a battery 250 (see FIG. 3), the grip electrode 213, a user's body, the contact electrode 31, and the battery 250 may be configured.

In the closed circuit, the contact electrode 31 and the grip electrode 213 may form different polarities, respectively. For example, the contact electrode 31 may form (+) polarity and the grip electrode 213 may form (−) polarity. As the contact electrode 31 forms (+) polarity, waste having a (−) polarity present on the surface or inside of the skin may be discharged to the outside of the skin by electrical attraction, and the discharged waste may be adsorbed to the contact electrode 31.

Although not shown in the drawings, according to an embodiment, an indicator (e.g., a light source) for notifying a power state or an operating state of the skin care device 1 may be provided at a prescribed position of the case 201.

The head 3 may include the contact electrode 31 in contact with a skin. The contact electrode 31 may form a prescribed polarity ((+) polarity) to discharge the (−)-polarity waste present on or inside the skin surface to the outside of the skin.

In some implementations, the contact electrode 31 may include a plurality of contact protrusions 312 (see FIG. 3). Accordingly, the contact electrode 31 may come in contact with the skin in the form of point contacts through a plurality of the contact protrusions 312.

For example, when the contact electrode 31 includes a plate, it may come in contact with the skin in the form of a surface contact. In this case, it may be advantageous in that a contact area with the skin is wider than that of a case of including a plurality of the contact protrusions 312. However, when the skin care device 1 is used for a skin (such as a scalp, etc.) at a part where hair or the like is present, a contact between the plate and the skin may not be effectively made due to the hair. In this case, there is a possibility that side effects such as hair damage and the like may occur as the waste inside the skin is not effectively eliminated, or the waste discharged to the outside remains on the hair and the like.

Accordingly, the contact electrode 31 according to the present embodiment may come in contact with the skin in the form of point contacts through a plurality of the contact protrusions 312, thereby more effectively eliminating waste inside the skin and minimizing the amount of waste remaining on the hair and the like.

The cap 4 may be detachably formed on the head 3. When the skin care device 1 is not used, the cap 4 may be mounted on the head 3, and in this case, the contact electrode 31 may not be exposed to the outside. That is, the cap 4 may prevent external dust, foreign substances, and bacteria from adsorbing to the contact electrode 31, thereby maintaining the cleanliness of the contact electrode 31.

Hereinafter, embodiments related to the structure and operation of the skin care device 1 will be described with reference to FIGS. 3 to 10.

FIG. 3 is one schematic cross-sectional diagram of a skin care device.

In the following drawings, the head 3 is defined as being positioned above the body 2. In addition, at least one button 211 is defined as being positioned in front of the grip electrode 213.

Referring to FIG. 3, an inner frame 221, an upper frame 222 connected to a topside of the inner frame 221, and a lower frame 223 connected to a bottom side of the inner frame 221 may be provided within the body 2.

The inner frame 221 may be elongated in a top-bottom direction based on a shape of the body 2. Various components inside the body 2 may be mounted on or connected to the inner frame 221. For example, a main board 231, a battery 250 and the like may be connected to or mounted on the inner frame 221.

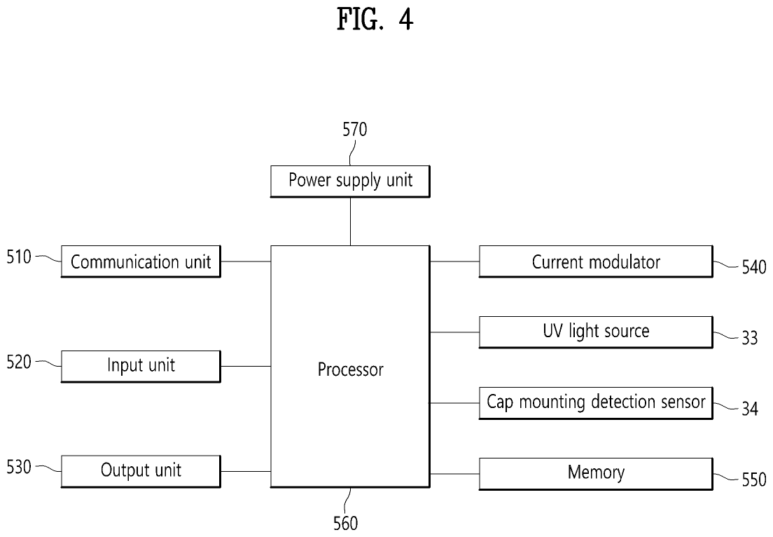

The main board 231 may be connected to a front side of the inner frame 221. Control components such as a processor, a memory, a communication unit and the like may be provided to the main board 231. Contents related to the control components will be described in more detail later with reference to FIG. 4.

In addition, at least one or more connecting parts 234a and 234b connected to the at least one button 211 may be provided to the main board 231. For example, the connecting parts 234a and 234b may be fastened to the front surface of the main board 231.

When one of the at least one or more buttons 211 is manipulated, the processor 560 may obtain a signal from the connecting part corresponding to the manipulated button, thereby detecting the manipulation of the button.

The battery 250 may be located in rear of the main board 231. The battery 250 may supply power to each of the components of the skin care device 1.

Meanwhile, the battery 250 may be electrically connected to each of the grip electrode 213 and the contact electrode 31 to form a closed circuit. For example, the battery 250 may be electrically connected to the grip electrode 213 through a conductive plate 251, and may be electrically connected to the contact electrode 31 through the main board 231 and a wire 233.

The upper frame 222 may be connected to a top side of the inner frame 221. Specifically, the upper frame 222 may be connected between a head frame 32 of the head 3 and the inner frame 221 to fix the head frame 32 to the inner frame 221.

The lower frame 223 may be connected to a bottom side of the inner frame 221. A sub-board 232, a power supply terminal 235, a microphone, and/or a speaker 240 may be connected to the lower frame 223.

The sub-board 232 may be connected between the power supply terminal 235 and the battery 250. The sub-board 232 may be provided with a processor (e.g., micom, integrated circuit, etc.) that performs a control operation related to charging of the battery 250.

The power supply terminal 235 is provided to a bottom side of the body 2, and may be connected to an external power supply device to supply power to the battery 250. According to an embodiment, when power of the battery 250 is charged to a prescribed level or higher, the processor of the sub-board 232 may control to stop supplying power to the battery 250, thereby preventing abnormality of the battery 250 due to overcharge.

According to an embodiment, the power supply terminal 235 may function as a communication connection terminal for connecting communication between an external device and the skin care device 1. In this case, the skin care device 1 may transmit and receive data to and from the external device connected through the communication connection terminal.

The microphone and/or speaker 240 may be disposed at a position corresponding to the hole 212 formed in the case 21. The microphone and/or speaker 240 may communicate with the outside through the hole 212 to provide sound to the outside or obtain voice from the outside.

Referring still to FIG. 3, the head 3 may include a contact electrode 31, a head frame 32, at least one ultraviolet light source 33, and a cap mounting detection sensor 34.

Components 31, 33 and 34 provided to the head 3 may be connected to or mounted on the head frame 32. The head frame 32 may be supported with respect to the inner frame 221 by being fastened to the upper frame 222. According to an embodiment, the head frame 32 and the upper frame 222 may be integrally formed.

The contact electrode 31 may be fastened to the case 21 or the head frame 32 to be positioned above the head frame 32. As the contact electrode 31 is fastened, the components in the body 2 and the head 3 may avoid being exposed to the outside.

The contact electrode 31 may come in contact with a user's skin to eliminate waste such as fine dust present on the surface or inside of the skin. The contact electrode 31 may be implemented with a conductive metal such as SUS, aluminum, etc.

For example, the contact electrode 31 may include a fastening part 311 fastened to the head 3 (specifically, the case 21 of the head part) or the head frame 32.

The fastening part 311 may have a roughly cylindrical shape, and an opening may be formed in a bottom side of the fastening part 311. As the opening is formed, a receiving space for receiving at least one ultraviolet (UV) light source 33, the cap mounting detection sensor 34 and the like therein may be formed within the fastening part 311.

A plurality of contact protrusions 312 may be formed on a top surface of the fastening part 311. Each of a plurality of the contact protrusions 312 may be formed in a manner of protruding at a prescribed height from the top surface of the fastening part 311. A plurality of the contact protrusions 312 correspond to a contact means directly coming in contact with a user's skin (e.g., scalp, etc.).

According to an embodiment, each of a plurality of the contact protrusions 312 may have a shape in which the width thereof decreases as it becomes farther from the top surface of the fastening part 311. That is, the contact shape of each of a plurality of the contact protrusions 312 may correspond to a point contact or a surface contact having a contact area equal to or smaller than a prescribed area. Accordingly, each of a plurality of the contact protrusions 312 may more effectively come in contact with a scalp even when the skin care device 1 is used for the scalp in which hair or the like exists.

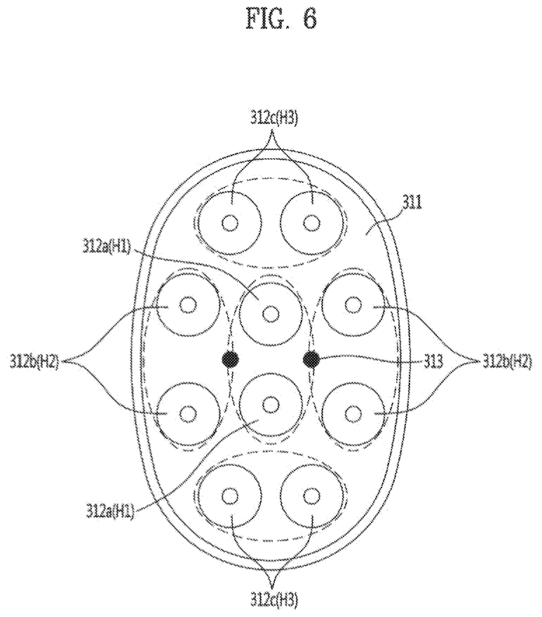

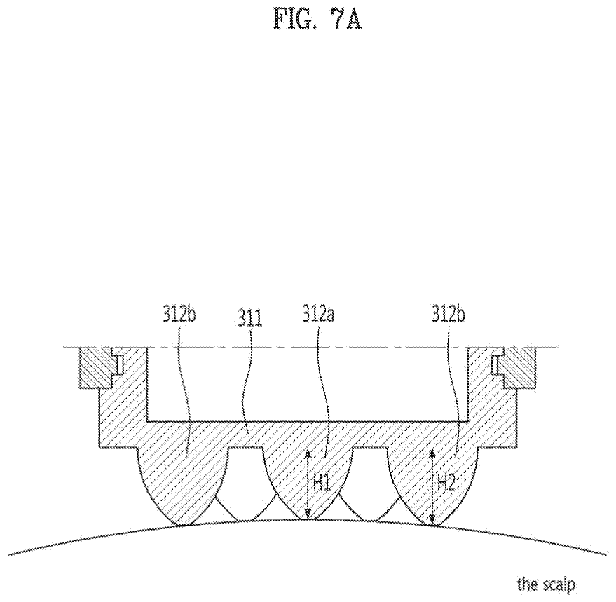

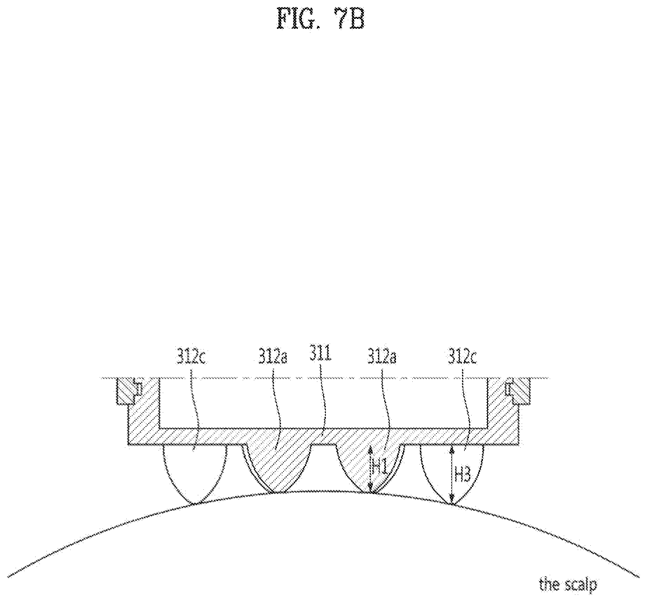

In some implementations, heights of a plurality of the contact protrusions 312 may be different from each other. The relevant description will be made later with reference to FIGS. 6 to 7B.

The at least one ultraviolet light source 33 may be provided on a top surface of the head frame 32. As the contact electrode 31 is fastened to the case 21 or the head frame 32, the at least one ultraviolet light source 33 may be received in a space between the contact electrode 31 and the head frame 32.

The at least one ultraviolet light source 33 may be disposed to face a direction corresponding to a protruding direction of the contact protrusion while received in the space. The at least one ultraviolet light source 33 may emit ultraviolet light for a prescribed time when the cap 4 is mounted on the head 3.

At least one hole 313 for irradiating light emitted from the at least one ultraviolet light source 33 to an inner surface of the cap 4 may be formed in a top surface of the fastening part 311 of the contact electrode 31. The at least one hole 313 may be formed to correspond to each of the at least one or more ultraviolet light sources 33.

According to an embodiment, the at least one hole 313 may be provided with a light transmitting part (e.g., glass made of a transparent material), or may be further provided with a lens for enlarging an irradiation area of the ultraviolet light.

The ultraviolet light irradiated through the at least one hole 313 may be irradiated to the top side of the contact electrode 31 as it is reflected by a light reflection part 402 formed on an inner surface of the cap 4. Accordingly, the top surface of the fastening part 311 of the contact electrode 31 and a plurality of the contact protrusions 312 are sterilized, so that the cleanliness of the contact electrode 31 can be maintained.

The at least one ultraviolet light source 33 may be implemented with a UV-LED that emits ultraviolet light of a UV-C band, by which the present disclosure is non-limited.

That is, according to an embodiment of the present disclosure, since a configuration (e.g., the ultraviolet light source 33) for sterilization of the contact electrode 31 may be provided to the head 3, the cap 4 may not be provided with an ultraviolet light source and a configuration for supplying power. Accordingly, the structure of the cap 4 may be simplified, and since other additional devices such as a cradle for sterilizing the contact electrode 31 are not required, the configuration of the entire package of the skin care device 1 may be simplified.

On the other hand, when ultraviolet light is emitted while the cap 4 is not mounted, there is a concern that the ultraviolet light is irradiated to user's skin or eyes and causes various diseases.

Therefore, the head 3 may be provided with a cap mounting detection sensor 34 for detecting that the cap 4 is mounted on the head 3. The processor 560 may detect whether the cap 4 is mounted through the cap mounting detection sensor 34, and may control the at least one ultraviolet light source 33 to emit ultraviolet light for a prescribed time when the cap 4 is mounted.

The cap mounting detection sensor 34 may be disposed on a top surface of the head frame 32 and may be received in a space between the contact electrode 31 and the head frame 32. Yet, since the disposed position of the cap mounting detection sensor 34 is not limited thereto, the cap mounting detection sensor 34 may be disposed at various positions for sensing that the cap 4 is mounted on the head 3.

For example, the cap mounting detection sensor 34 may be implemented with a hall sensor. In this case, a magnet 403 may be provided to the cap 4.

Contents related to the cap mounting detection sensor 34 will be described later with reference to FIG. 4.

The cap 4 may be mounted on the head 3 when the skin care device 1 is not used, thereby shielding the contact electrode 31 from the outside. Accordingly, dust, foreign substances, bacteria, etc. may be prevented from coming in contact with or adsorbing to the contact electrode 31, thereby preventing deterioration in cleanliness.

For example, the cap 4 may include a cap body 401 and a light reflecting part 402 formed on an inner surface of the cap body 401.

The cap body 401 may be detachably formed on a top side of the head 3. The cap body 401 may be generally formed in a dome shape (e.g., a dome shape having a planar top surface). Accordingly, a plurality of the contact protrusions 312 may be received in a space inside the cap 4 when the cap 4 is mounted.

In some implementations, the cap body 401 may contain a material that does not transmit ultraviolet rays or has an ultraviolet transmittance less than a prescribed value. Accordingly, side effects or damage caused by transmission of ultraviolet light emitted from the at least one ultraviolet light source 33 to a human body and the like may be prevented. According to an embodiment, when the light reflecting part 402 is provided on the entire inner surface of the cap body 401, the cap body 401 may include various materials irrespective of the ultraviolet transmittance.

The light reflecting part 402 reflecting ultraviolet light may be provided on an inner surface of the cap body 401. The light reflecting part 402 may be formed on a surface of the inner surface of the cap body 401, which confronts the contact electrode 31, but is not limited thereto, and may also be formed on a lateral surface of the inner surface of the cap body 401.

The light reflecting part 402 may be implemented with a mirror attached to the inner surface of the cap body 401, but is not limited thereto, and may include silver, aluminum, or the like coated on the inner surface or attached thereto in a film form.

According to an embodiment, when the cap mounting detection sensor 34 is implemented with a hall sensor, the cap 4 may further include a magnet 403. The magnet 403 may be mounted at a prescribed position of the cap body 401. For example, assuming that a distance at which a magnetic field of the magnet 403 is formed is a first distance, the magnet 403 may be mounted at a position less than or equal to the first distance from the cap mounting detection sensor 34 when the cap 4 is mounted on the head 3.

FIG. 4 is a block diagram schematically showing a control configuration of a skin care device.

Referring to FIG. 4, a skin care device 1 may include a communication unit 510, an input unit 520, an output unit 530, a current modulator 540, a memory 550, a processor 560, and a power supply unit 570. The control components illustrated in FIG. 4 are examples for convenience of description, and the skin care device 1 may include more or less components than the control components illustrated in FIG. 4.

The communication unit 510 may connect the skin care device 1 to an electronic device such as a smartphone, a tablet PC, a PC, a server, etc. The skin care device 1 may transmit and receive data to and from the electronic device through the communication unit 510. For example, the communication unit 510 may include at least one communication module (communication interface) that supports various wired/wireless communication systems.

The at least one communication module may support a wired communication system such as a Universal Serial Bus (USB), a Universal Synchronous Receiver-Transmitter (UART), an RS232, etc., or a wireless communication system such as Wi-Fi, Bluetooth, NFC, etc.

The at least one communication module may be mounted on the main board 231 or the sub-board 232, but is not limited thereto.

The input unit 520 may receive various types of user inputs related to the operation of the skin care device 1. For example, the input unit 520 may include the at least one button 211, the microphone or the like described above. The at least one button 211 may receive an input of a physical type (such as a push) from a user, and the microphone may receive an input of a voice type from the user. The input may include various commands such as power on/off, operation mode setting and the like of the skin care device 1.

The output unit 530 may provide various informations such as an operation state of the skin care device 1 and the like to a user. For example, the output unit 530 may include various visual/auditory/tactile output means such as a speaker, a light source (indicator), a display, a vibration motor (haptic module), and the like.

When a closed circuit connecting the battery 250, the grip electrode 213, a user's body, the contact electrode 31, and the battery 250 is configured, the current modulator 540 may adjust characteristics (e.g., waveform, size, frequency, etc.) of a current applied to the closed circuit under control of the processor 560.

The memory 550 may store various types of data such as control data for controlling operations of the components included in the skin care device 1, data received through the communication unit 510, data for performing an operation based on an input acquired through the input unit 520, and the like.

The memory 550 may include various storage devices such as ROM, RAM, EEPROM, flash drive and the like in hardware.

The processor 560 may control the overall operation of the skin care device 1. For example, the processor 560 may include at least one microcomputer, an integrated circuit, an Application Specific Integrated Circuit (ASIC), an Application Processor (AP), and the like.

When the skin care function is executed, the processor 560 may control the current modulator 540 to apply a current having a prescribed characteristic to a skin.

Meanwhile, the processor 560 may detect the mounting of the cap 4 based on a sensing value of the cap mounting detection sensor 34, and may drive the at least one ultraviolet light source 33 based on the detection result for a prescribed time.

As described above, the cap mounting detection sensor 34 may be implemented with a hall sensor. As the cap 4 is mounted on the head 3, when the cap mounting detection sensor 34 and the magnet 403 approach each other within a prescribed distance, a potential difference (i.e., a sensing value) may be formed in the cap mounting detecting sensor 34 by a magnetic field generated from the magnet 403. The processor 560 may detect the mounting of the cap 4 based on the potential difference and drive the at least one ultraviolet light source 33 to perform sterilization on the contact electrode 31.

The power supply unit 570 may supply power for operation of components provided to the skin care device 1. For example, the power supply unit 570 may include the battery 250 described above in FIG. 3.

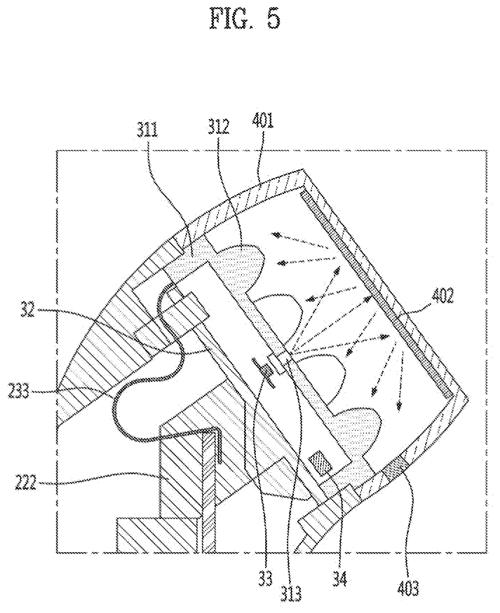

Hereinafter, embodiments related to the skin care device 1 will be described in detail with reference to FIGS. 5 to 10.

FIG. 5 is a diagram to describe an embodiment related to sterilization of a contact electrode provided to a head of a skin care device.

Referring to FIG. 5, as described above in FIG. 3, the processor 560 may detect that the cap 4 is mounted on the head 3 through the cap mounting detection sensor 34 and control the at least one ultraviolet light source 33 to emit ultraviolet light for a prescribed time.

The at least one ultraviolet light source 33 may be received between the fastening part 311 of the contact electrode 31 and the head frame 32. The ultraviolet light emitted from the at least one ultraviolet light source 33 may be irradiated over the fastening part 311 through the at least one hole 313. According to an embodiment, the at least one hole 313 may be further provided with a lens for increasing an irradiation area of the ultraviolet light.

The ultraviolet light irradiated over the fastening part 311 may be reflected by the light reflecting part 402 formed on the inner surface of the cap body 401, and the reflected ultraviolet light may be irradiated to the top surface of the fastening part 311 and a plurality of fastening protrusions 312.

On the other hand, as the contact electrode 31 is implemented with a material such as SUS or the like, a portion of the light irradiated to the top surface of the fastening part 311 and the fastening protrusions 312 may be reflected again by the light reflecting part 402. That is, as the ultraviolet light is reflected multiple times, uniform sterilization of various areas of the contact electrode 31 may be performed.

According to an embodiment, a surface of the light reflecting part 402 may be implemented as a non-planar surface. For example, the surface of the light reflecting part 402 may form an irregular surface to reflect ultraviolet light to various positions. That is, as shown in FIG. 5, when the ultraviolet light source 33 is disposed to be close to the center of the contact electrode 31, the light reflecting part 402 is configured to sufficiently irradiate ultraviolet light to an edge area of the contact electrode 31, thereby providing a sufficient sterilization effect on the entire area of the contact electrode 31.

Although FIG. 5 illustrates that the light reflecting part 402 is formed only on the surface of the inner surface of the cap body 401, which confronts the contact electrode 31, the light reflecting part according to an embodiment may be formed on the entire inner surface of the cap body 401.

The processor 560 drives the at least one ultraviolet light source 33 for a prescribed time to sterilize microorganisms or bacteria present on the contact electrode 31, and then stops driving the at least one ultraviolet light source 33, thereby preventing unnecessary power consumption due to the continuous driving of the ultraviolet light source 33.

That is, according to the embodiment shown in FIG. 5, the skin care device 1 includes the at least one ultraviolet light source 33 to provide a function of sterilization on the contact electrode 31, thereby maintaining cleanliness of the contact electrode 31 in direct contact with a skin. Accordingly, it is possible to effectively prevent side effects such as deterioration of the skin condition due to bacteria remaining on the contact electrode 31.

In addition, as the light reflecting part 402 is formed in the cap 4, the ultraviolet light source 33 may be provided to the head 3. That is, since the cap 4 may not be provided with an ultraviolet light source and a configuration for supplying power, the structure of the cap 4 may be simplified and other additional devices such as a cradle for sterilizing the contact electrode 31 and the like are not required. As a result, the configuration of the entire package of the skin care device 1 may be simplified.

FIGS. 6 to 7B are diagrams to describe an embodiment related to a plurality of contact protrusions included in a contact electrode.

Referring to FIG. 6, as described in FIG. 3, the contact electrode 31 may include a plurality of contact protrusions 312a to 312c protruding from the top surface of the fastening part 311. As a plurality of the contact protrusions 312a to 312c are formed, the contact between the skin (scalp) and the contact electrode 31 may be facilitated when the skin management device 1 is used for the scalp where hair and the like exist.

On the other hand, according to an embodiment of the present disclosure, heights H1 to H3 of a plurality of the contact protrusions 312a to 312c may be different from each other depending on a distance spaced apart from the center of the fastening part 311.

Specifically, the longer the distance spaced apart from the center of the fastening part 311 gets, the higher each of the heights H1 to H3 of a plurality of the contact protrusions 312a to 312c becomes.

Like the embodiment of FIG. 6, when the top surface of the fastening part 311 has an oval shape, a plurality of the contact protrusions 312a to 312c may include first contact protrusions 312a spaced apart from the center of the fastening part 311 by a first distance, second contact protrusions 312b spaced apart from the center by a second distance longer than the first distance, and third contact protrusions 312c spaced apart from the center by a third distance longer than the second distance.

In this case, the second height H2 of the second contact protrusions 312b may be greater than or equal to the first height H1 of the first contact protrusions 312a, and the third height H3 of the third contact protrusions 312c may be greater than or equal to the second height H2.

In this regard, referring to FIG. 7A and FIG. to 7B, since a head shape of a human body has an approximately spherical shape, a scalp may be formed to be approximately rounded along the head shape. In addition, most of the skins of a face, arms, and legs are formed to be rounded according to the skeleton of the human body.

Namely, when a plurality of the contact protrusions 312a to 312c have the same height, the first contact protrusions 312a close to the center of the fastening part 311 may smoothly come in contact with the skin of a scalp and the like, but the second contact protrusions 312b and the third contact protrusions 312c may not effectively come in contact with the scalp.

According to the present embodiment, as each of the contact protrusions 312a to 312c is formed to have a height increasing when getting far away from the center of the fastening part 311, all the contact protrusions 312a to 312c may effectively come in contact with a skin when coming in contact with the skin having a curved surface such as a scalp. Accordingly, the care (cleansing) effect provided by the skin care device 1 may be maximized.

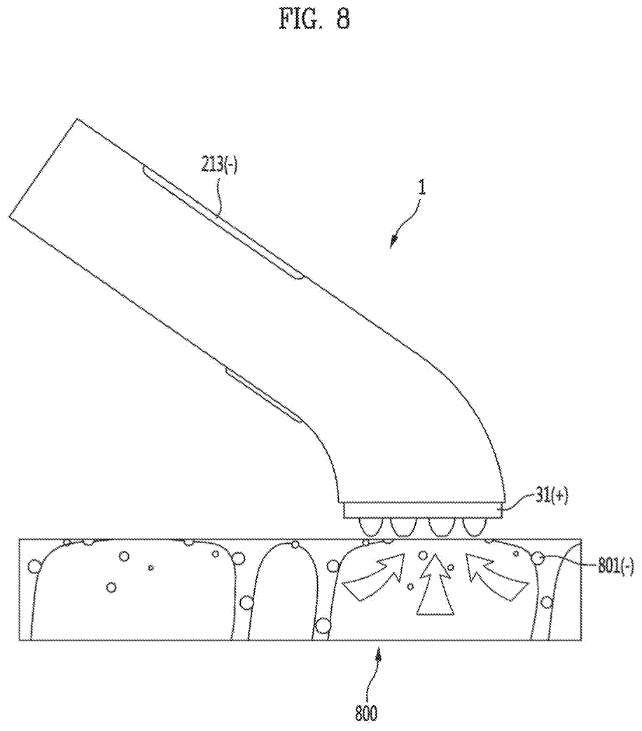

FIG. 8 is a diagram to describe the operation principle of a skin care device according to one embodiment of the present disclosure.

Referring to FIG. 8, as described in FIG. 3, a closed circuit may be formed as a user holds the case 21 by hand and contacts the contact electrode 31 with a skin 800.

Meanwhile, a waste 801 such as fine dust present on or inside a skin surface (e.g., extracellular fluid, intercellular epilepsy, etc.) has (−) polarity, and thus the contact electrode 31 may form (+) polarity to discharge the waste 801 from the skin. That is, the processor 560 may control the current modulator 540 to enable the contact electrode 31 to form (+) polarity.

However, simply forming the (+) polarity of the contact electrode 31 does not easily discharge the waste 801 present inside the skin. That is, in order to effectively discharge the waste 801, the characteristics (size, frequency, pattern, etc.) of the current provided by the current modulator 540 should be determined.

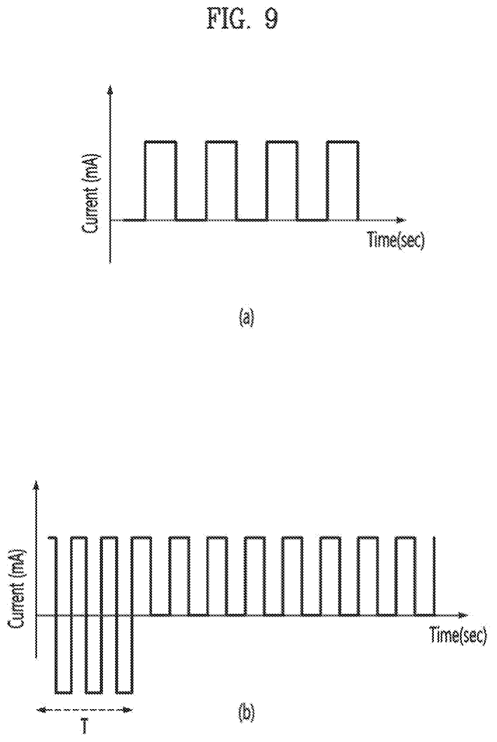

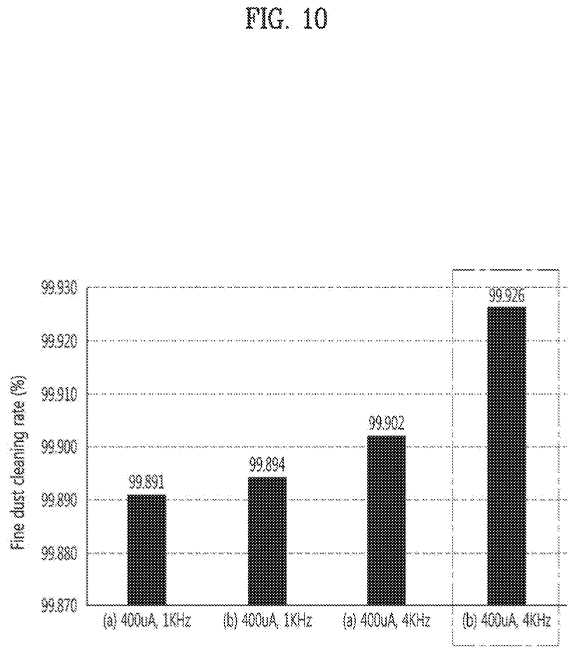

FIG. 9 and FIG. 10 are diagrams to describe a cleansing effect according to the characteristic of a current applied to a contact electrode of a skin care device.

Referring to FIG. 9 and FIG. 10, the magnitude of the current among the characteristics of the current may be related to the adsorption power to the waste or the location (depth in the skin) of the absorbable waste.

Among the characteristics of the current, the frequency of the current may be related to the material diffusion rate (or ion transfer rate within a cell) of the extracellular fluid. That is, when a current of a frequency that accelerates the diffusion of substances in the extracellular fluid is applied, the waste moves more quickly from the inside of the skin to the outside, so the waste can be effectively eliminated. As shown in the experimental data of FIG. 10, it may be observed that when the frequency is 4 KHz, the cleaning rate of fine dust is higher than when the frequency is 1 KHz. That is, when a current having a frequency of 4 KHz is applied, the diffusion rate of the substance of the extracellular fluid is accelerated, and waste may be more effectively eliminated. Based on this, the current modulator 540 may set a frequency of the current in a range between about 3 KHz and 10 KHz. More preferably, the current modulator 540 may set the frequency of the current to about 4 KHz.

Meanwhile, referring to FIG. 9 (a) and FIG. 9 (b) with respect to the pattern of the current among the characteristics of the current, the processor 560 may control the current modulator 540 to provide an alternating current to the contact electrode 31. In this case, in order for the contact electrode 31 to adsorb the waste having (−) polarity present in the skin, the current modulator 540 may adjust the current pattern so that the contact electrode 31 generally forms (+) polarity.

In case of the embodiment shown in FIG. 9 (a), the current modulator 540 may adjust the current pattern so that the contact electrode 31 does not form (−) polarity.

Meanwhile, in case of the embodiment shown in FIG. 9 (b), the current modulator 540 may adjust the current pattern so that the contact electrode 31 alternately forms (+) polarity and (−) polarity for a prescribed time T (or a prescribed period) preset from a time point of contacting the contact electrode 31 with a skin while the skin care device 1 is driven. For example, for the prescribed time T, (−) polarity may be formed in the contact electrode 31 for at least one period.

Referring to FIG. 10, it may be confirmed that a fine dust cleaning rate when a current of a pattern according to FIG. 9 (b) is applied is higher than when a current of a pattern according to FIG. 9 (a) is applied.

When the contact electrode 31 alternately forms the (+) polarity and the (−) polarity for the prescribed time T as shown in FIG. 9 (b), electrical vibration is applied to the inside of the cell (extracellular fluid and/or intercellular lipids), thereby promoting material movement activation of the cell. Accordingly, since the movement of wastes present in the cell is promoted, the wastes may be more effectively discharged to the outside of the skin.

Namely, the skin care device 1 may be efficiently driven according to the current characteristics capable of maximizing elimination of wastes inside a skin.

The description above is merely illustrative of the technical idea of the present disclosure, and various modifications and changes may be made by those skilled in the art without departing from the main characteristics of the present disclosure.

Therefore, the embodiments disclosed in the present disclosure are not intended to limit the technical idea of the present disclosure but to illustrate the present disclosure, and the scope of the technical idea of the present disclosure is not limited by the embodiments.

The scope of the present disclosure should be construed as being covered by the scope of the appended claims, and all technical ideas falling within the scope of the claims should be construed as being included in the scope of the present disclosure.

1. A skin care device, comprising:
a body including a battery and a grip electrode electrically connected to the battery; and
a head connected to one end of the body,
wherein the head comprises:
a contact electrode electrically connected to the battery and configured to come in contact with a skin of a user; and
at least one ultraviolet light source disposed inside the head to face the contact electrode, and
wherein the contact electrode comprising comprises:
a fastening part fastened to the head and having at least one hole formed therein to correspond to the at least one ultraviolet light source; and
a plurality of contact protrusions protruding from a top surface of the fastening part.

2. The skin care device of claim 1, further comprising a cap detachably attached to the head,
wherein the cap comprises:
a cap body in a dome shape; and
a light reflecting part formed on at least one portion of an inner surface of the cap body.

3. The skin care device of claim 2, wherein the light reflecting part is formed on a surface confronting the contact electrode in the inner surface of the cap body.

4. The skin care device of claim 3, wherein a surface of the light reflecting part forms a non-planar surface or an irregular surface.

5. The skin care device of claim 2, wherein the head further comprises a cap mounting detection sensor used to detect whether the cap is mounted on the head.

6. The skin care device of claim 5, further comprising a processor configured to drive the at least one ultraviolet light source for a preset time in response to detecting that the cap is mounted on the head through the cap mounting detection sensor.

7. The skin care device of claim 5, wherein the cap mounting detection sensor comprises a hall sensor, and
wherein the cap further comprises a magnet.

8. The skin care device of claim 1, wherein the plurality of contact protrusions form different protrusion heights based on distances spaced apart from a center of a top surface of the fastening part, respectively.

9. The skin care device of claim 8, wherein the plurality of contact protrusions comprise:
a first contact protrusion spaced apart from the center by a first distance; and
a second contact protrusion spaced apart from the center by a second distance longer than the first distance, and
wherein a protrusion height of the second contact protrusion is equal to or greater than that of the first contact protrusion.

10. The skin care device of claim 1, wherein the body further comprises a board disposed to correspond to a length direction of the body, and
wherein a protrusion direction of each of the plurality of contact protrusions forms an inclination at a prescribed angle with respect to a length direction of the board.

11. The skin care device of claim 10, wherein the prescribed angle is a right angle or an obtuse angle.

12. The skin care device of claim 1, wherein the body further comprises a processor and a current modulator, and
wherein when the contact electrode comes in contact with the skin of the user while the skin care device is driven, the processor controls the current modulator to form a positive (+) polarity in the contact electrode.

13. The skin care device of claim 12, wherein the processor controls the current modulator to alternately form the positive (+) polarity and a negative (−) polarity in the contact electrode for a preset time from a time point of contacting the contact electrode with the skin of the user.

14. The skin care device of claim 12, wherein the processor controls the current modulator to form a negative (−) polarity in the contact electrode for at least one period for a preset time from a time point of contacting the contact electrode with the skin of the user.

15. A method of controlling a skin care device including a contact electrode configured to come in contact with a skin of a user, a battery electrically connected to the contact electrode, a current modulator adjusting a current applied to the contact electrode, and a processor, the method comprising:
based on contacting the contact electrode with the skin of the user, controlling the current modulator to apply a current according to a first pattern to the contact electrode for a preset time from a contact initiation time point; and
after a lapse of the preset time, controlling the current modulator to apply a current according to a second pattern different from the first pattern to the contact electrode,
wherein when the current according to the first pattern is applied, a negative (−) polarity is formed in the contact electrode for at least one period of the preset time.

16. The method of claim 15, wherein when the current according to the first pattern is applied, a positive (+) polarity and the negative (−) polarity are alternately formed in the contact electrode.

17. The method of claim 15, wherein when the current according to the second pattern is applied, the negative (−) polarity is not formed in the contact electrode.

18. The method of claim 15, wherein a current having a frequency between about 3 KHz and about 10 KHz is applied to the contact electrode.

19. The method of claim 15, wherein the skin are device further comprises at least one ultraviolet light source and a cap mounting detection sensor disposed within a head,
wherein the contact electrode comprises at least one hole corresponding to the at least one ultraviolet light source, and
wherein the method further comprises detecting that the cap is mounted using the cap mounting detection sensor, and based on detecting that the cap is mounted, driving the at least one ultraviolet light source for another preset time.

20. A skin care device, comprising:
a body configured to enable a user to grip the body; and
a head connected to one end of the body,
wherein the head comprises a contact electrode configured to come in contact with a skin of the user,
wherein the contact electrode comprises a plurality of contact protrusions, and
wherein the contact electrode is configured to be applied with at a current of at least one polarity at a frequency of between about 3 KHz and about 10 KHz when the contact electrode is in contact with the skin of the user to clean dust from the skin of the user.
